(19) United States
(12) Patent Application Publication
(10) Pub. No.: US 2005/0202088 A1
(43) Pub. Date: Sep. 15, 2005

(54) Pharmaceutical composition having a delayed active substance release, and method for the preparation thereof

(76) Inventors: Franke Hanshermann, Tangstedt (DE); Peter Lennartz, Hamburg (DE); Jorn Raimer, Hamburg (DE)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/516,268

(22) PCT Filed: May 15, 2003
(86) PCT No.: PCT/EP03/05115

§ 371 Date: May 27, 2005

(30) Foreign Application Priority Data
May 31, 2002 (DE) 102 24 170.8

(51) Int. Cl. A61K 9/24

(52) U.S. Cl. 424/471; 264/109

(57) ABSTRACT

A pharmaceutical composition with sustained drug release is described which is obtainable by a specific compaction process wherein organic solvents and water are not required and which is preferably in form of single drug compartments or which disintegrates into such compartments upon contact with aqueous media.

The invention relates to a pharmaceutical composition with sustained drug release, which is present in particular in the form of a multiple unit dosage form, and a process for its preparation.

In the development of pharmaceutical compositions with sustained release of the active ingredient, so-called multiple unit dosage forms (MUDs) are to be preferred over single unit dosage forms (SUDs) because of various advantages. The multiple unit dosage forms represent orally administered compositions which are either already present in the form of a plurality of functional drug compartments or decompose after intake into such functional drug compartments. They thus make possible an optimum therapy from biopharmaceutical aspects. For example, the release of the active ingredient from them is largely independent of the stomach fill level of the patient and a very uniform release of the active ingredient is achieved even with different patients. Finally the phenomenon of so-called “dose dumping” is avoided (cf. J. Butler et al., Pharm. Technol. 1998, pages 122 to 138). By “dose dumping” is meant the uncontrolled, rapid release of all or a large part of the drug dose from a dosage form which is actually intended to release the drug in a sustained and controlled manner.

The drug compartments of a multiple unit dosage form ideally measure 2 mm at most in every direction in space. Only compartments of this order of magnitude achieve the desired optimum in-vivo release pattern with only a very small influence being exerted by the type and quantity of food eaten.

Multiple unit dosage forms can be e.g. granulates and minitablets with particle sizes of in particular 2 mm at most in every direction in space or also tablets which decompose after intake to particles containing drug and measuring in particular 2 mm at most in every direction in space.

Multiple unit dosage forms releasing the drug in a sustained manner are usually prepared by pelletizing, mini- or microtabletting or wet granulation. The drug is present in a sustained-release matrix or the drug compartments are provided with a sustained-release film. However, solvents, such as organic solvents and water, are necessary for all these process steps. However, organic solvents are ecologically and toxicologically unacceptable. The use of water can also mean that the finished dosage form does not have the desired stability, for which reason costly and uneconomical drying steps are necessary as a rule. Also, the use of water can already lead to stability problems during preparation.

A known process for the preparation of granulates without solvents is forced compression, also called compaction, and subsequent crushing of the compressed solids. The aim of this variant, also called dry granulation, is as a rule to improve the flowability or increase the relative density of the powdery materials used. The drug can be compressed, between two rollers, alone or together with excipients, to produce a strand, also called a ribbon. This strand is then further crushed into a granulated material which has a clearly improved flowability compared with the powder mixture used.

Briquetting represents a further possibility for the dry preparation of granulated materials. During this process, the active ingredient is tabletted alone or together with excipients into large briquettes which are then further crushed to the desired size.

The aim of all these dry granulation processes is to compact the substances used in order to improve their flow properties or also improve their compressibility, e.g. during a subsequent tabletting.

In WO 00/08092, the preparation of polyacrylic acid granulates by compression between two rollers is described. The granulated material prepared is then mixed with theophylline as drug and further tabletting excipients and pressed into tablets with sustained release of the active ingredient.

Furthermore, microtablets with a high theophylline content in a polymer matrix were prepared by H. Rey et al. in Drug Development and Industrial Pharmacy 26, 21-26 (2000). The microtablets displayed a sustained release of the active ingredient and were prepared from granulate in a tablet press. It was also established that the compression pressure used during the tabletting had no influence whatsoever on the release behaviour of the active ingredient.

Paul J. Scheskey et al. describe in Pharm. Technol. Eur. 11/11, 18-35 (1999) the compaction of mixtures of theophylline with hydroxypropylmethyl cellulose using rollers. During the compaction the roller temperature was kept at 22° C. with the help of a circulating coolant. The authors tested the influence of various parameters of the compaction process and found that the compaction pressure used had a negligible influence on the release behaviour of drug from corresponding tablets.

A similar result was achieved by Paul J. Scheskey et al. in Pharmaceutical Technology 18, 132-150 (1994) when examining sustained-release tablets with niacinamide as drug and methylcellulose or hydroxypropylmethyl cellulose. The granulate used to prepare the tablets was prepared by compaction using rollers at different pressures. Although a better flow behaviour was able to be achieved, no relationship between roller pressure used and rate of release of the active ingredient was found.

Furthermore, the process parameters during the dry granulation of theophylline formulations with sustained release of active ingredient were examined by P. Scheskey et. al. in Pharmaceutical Technology 24, 30-52 (2000) with the aim of transferring these from laboratory scale to industrial scale. Compaction forces per linear length unit of the roller width of 2.8, 3.1 and 3.4 t/inch were used, which in SI units corresponds to 10.8 kN/cm, 12.0 kN/cm and 13.2 kN/cm, respectively.

All in all the above publications show that the force exerted by the rollers during the compaction process has a negligible influence, or none whatsoever, on the release profile of sustained-release dosage forms. In every case, the compaction was used to influence physical solids parameters, but not to achieve a controlled release.

At most, it was found in the case of roller compaction of a mixture of low-substituted hydroxypropylmethyl cellulose and acetaminophen that, when the compaction pressure was increased, the rate of release of acetaminophen actually increased (cf. Y. Kawashima et al. in Chem. Pharm. Bull. 41, 1827-1831 (1993)).

There is thus a need for a pharmaceutical composition with sustained drug release and a process for its preparation in which a granulation takes place without the help of organic solvents or water and which allows the preparation of compositions, in particular in the form of multiple unit dosage forms with excellent sustained-release effect despite just small quantities of sustained-release polymer.

This object is achieved by the process according to one of claims 1 to 15 and the pharmaceutical compositions according to claims 16 to 19.

Surprisingly, it was shown that by selecting a specific compaction force and adjusting a specific temperature in the rollers used for compacting, pharmaceutical compositions in the very form of multiple unit dosage forms can be obtained which have a sustained drug release.

The process according to the invention for the preparation of a pharmaceutical composition with sustained drug release is characterized in that
(a) a mixture is provided which contains drug and a polymer effecting sustained drug release,
(b) the mixture is compressed by passing it between two rollers which have a temperature of more than 40° C. and which exert a force in the range from more than 15 and up to 40 kN/cm of roller width on the mixture,
(c) the compressed mixture is reduced to the desired particle size and
(d) optionally the compressed and reduced mixture is further processed.

In step (a) a mixture of drug and polymer is provided with the polymer effecting a sustained drug release. As drugs there can be considered a whole range of substances, and in particular the drug is selected from one of the following groups:
alpha- or beta-adrenergics,
alpha- or beta-adrenolytics,
analgesics,
antirheumatics,
antiarthritics,
anticholinergics,
anticonvulsants,
antidepressants,
antiparkinson agents,
antipsychotics,
anxiolytics,
dopamine receptor agonists,
antimigraine agents
neuroleptics,
neuroprotectives,
nootropics,
non-steroid antirheumatics,
sedatives and hypnotics.

Preferred representatives of the above groups of active ingredients are:

For anticonvulsants in particular
10-hydroxycarbamazepine
3-methyl-5-phenylhydantoin
4-amino-3-hydroxybutyric acid
5-methyl-5-(3-phenanthryl)hydantoin
acetazolamide
acetyl pheneturide
albutoin
aloxidone
aminoglutethimide
aminopentamide
atrolactamide
beclemide
benzodiazepine
bromine and bromides
buramate
carbamazepine
clobazam
clomethiazole
clonazepam
decimemide
Dilantin
dimethadione
diphenylan
diphenylhydantoin
doxenitoin
eterobarb
ethadione
ethoin
ethosuximide
famotidine
felbamate
fluoresone
fosphenytoin
gabapentin,
hakoseride

L-5-hydroxytryptophan and salts derived therefrom and complex compounds and mixtures of these,
lamotrigine
levetiracetam
magnesium sulphate
mephenytoin
mephobarbital
metharbital
methetoin
methsuximide
narcobarbital
divalproex sodium
nimetazepam
nitrazepam
oxcarbazepine
paramethadione
phenacemide
phenetharbital
pheneturide
phenobarbital
phensuximide
phenylmethylbarbituric acid
phenytoin
phenytoin sodium
phethenylate sodium
pregabalin
primidone
progabide
reboxetine
remacemide
rufinamide
suclofenide
sulthiame
talampanel
tetrantoin
tiagabine
topiramate
trimethadione
valproate sodium
valproic acid
valpromide
vigabatrin
zonisamide

For antiparkinson agents in particular:
amantadine
benserazide
bietanautine
biperiden
budipine
cabergoline
carbidopa
deprenyl
dexetimide
diethazine
droxidopa
entacapone
ethopropazine
ethylbenzhydramine
lazabemide
levodopa
memantine
mofegiline
piroheptine
pramipexole
pridinol
prodipine
ropinirole
selegiline
talipexole
terguride
tiapride
tigloidine
tolcapone
trihexyphenidyl hydrochloride

For antipsychotics, neuroleptics and antidepressants in particular:
acetophenazine
alizapride
amantadine
amisulpride
aripiprazole
befloxatone
benperidol
benserazide
benzquinamide
bietanautine
biperiden
bromocriptine
bromperidol
budipine
buramate
butaperazine
butyrophenone
cabergoline
carbidopa
carbipromin
carphenazine
carpipramine
chlorproethazine
chlorpromazine
chlorprothixene
clocapramine
clomacran
clopenthixole
clospirazine
clothiapine
clozapine
cyamemazine
dexetimide
diethazine
dixyrazine
droperidol
entacapone
ethopropazine
ethylbenzhydramine
fluanisone
flupentixole
fluphenazine
fluspirilene
haloperidol
iloperidones
imiclopazine
lazabemide
levodopa
lisuride
melperone
memantine
mepazine
mesoridazine
methoxypromazine
metofenazate
mofegiline
molindone
moperone
mosapramine
nemonapride
olanzapine
opipramol
oxaflumazine
penfluridol
perazine
pergolide
pericyazine
perimethazine
perphenazine
phenothiazine
pimozide
pipamperone
piperacetazine
piroheptine
pramipexole
pridinol
prochlorperazine
prodipine
promazine
prothipendyl
quetiapine
remoxipride
retigabine
risperidone
ropinirole
rotigentin
selegiline
sertindole
spiperone
sulforidazine
sulpride
sultopride
talipexole
terguride
tetrabenazine
thiopropazate
thioproperazine
thioridazine
thiothixene
thioxanthene
timiperone
tolcapone
trifluoperazine
trifluperidol
triflupromazine
trihexyphenidyl hydrochloride
ziprasidone
zotepine

For dopamine receptor agonists in particular:
bromocripitine
fenoldopam
lisuride
naxagolide
pergolide

For neuroprotectives in particular:
β-alanine derivatives
dizocilpine
xaliproden

Combinations of drugs can also be used. It is particularly preferred that the drug is an anticonvulsant, an antidepressant, an antiparkinson agent, an antipsychotic, an anxiolytic or a dopamine receptor agonist.

Oxcarbazepine, valproic acid or its salts, sulthiame, carbamazepine, lamotrigine or levetiracetam are particularly preferred.

Polymers conventionally used for the sustained release of an active ingredient in pharmaceutical compositions come into consideration as polymers. Primarily there can be named here polymers or copolymers of acrylic acid or acrylic acid derivatives or of methacrylic acid and methacrylic acid derivatives, cellulose polymers, waxes or fats. Further suitable substances are polyvinylpyrrolidone (PVP), PVP derivatives, polyethylene glycol (PEG), PEG derivatives, starch, starch derivatives, polyvinylchloride, polyethylene, polyvinyl acetate, polyvinyl alcohol, celluloses such as ethylcellulose, methylcellulose, hydroxypropylmethyl cellulose (HPMC), HPMC derivatives, hydroxypropyl cellulose, cellulose acetate, ethylene-vinyl acetate-copolymer or polyvinyl acetate-crotonic acid-copolymer.

An acrylic acid or methacrylic acid polymer, an acrylic acid or methacrylic acid copolymer or a salt thereof is particularly preferably used as polymers. Mixtures of different substances can also be used as polymers.

Furthermore, excipients from the groups colorants, flow regulators, lubricants, dry binders, disintegrants and stabilizers can also be added to the drug and the polymer.

Conventional mixers or mills are used to prepare the mixture.

It is preferred that the mixture contains 5 to 90 and in particular 70 to 85 wt.-% of drug. The quantity of polymer in the mixture is preferably 2 to 50 and in particular 5 to 30 wt.-% of polymer.

The mixture is then compressed in step (b). The process is in particular that the mixture is transported by suitable conveying means, such as e.g. a conveyor worm, to the two rollers turning in opposite directions and passed between these two rollers. The rollers have a temperature greater than 40° C. and for this purpose they are usually provided with a thermostatting apparatus. It is preferred that the temperature of the rollers does not exceed 100° C. The temperature of the rollers is particularly preferably in the range from 70 to 90° C.

It is also important that the rollers exert a force on the mixture which is in the range from 15 and up to 40 kN/cm of roller width. This force is set as a rule by measuring the deformation at the engine frame using wire resistance strain gauges or by measuring pressures in the hydraulics area and also calibrating the measuring apparatus using force-measurement heads which are mounted between the rollers. The force is then controlled via SPC. In this connection, roller width means the length of the direct connection line between the roller edges. With this definition used according to the invention, there are no problems in determining the roller width, as can occur e.g. in the case of rollers with uneven surfaces.

It was shown that the rollers particularly preferably exert a force on the mixture in the range from 18 to 23 kN/cm of roller width.

Customary roller compacters which make it possible to adjust the force exerted on the mixture to be compacted in the range according to the invention and to heat the rollers to the required temperatures are suitable for the process according to the invention. Suitable are for example 3-W-Polygran machines from Gerteis Maschinen+Processengineering AG, Jona, Switzerland.

As a result of the compression of the mixture by the two rollers, the mixture usually assumes the form of a strand which is also called a ribbon.

In step (c), the compressed mixture is reduced to the desired particle size with particle sizes of 50 to 1000 μm in every direction in space being preferred. The reducing to the desired particle size is carried out with customary means, such as screening, milling or breaking.

The compressed and reduced mixture present after this step already represents a multiple unit dosage form, i.e. a granulated material which consists of functional drug compartments.

In step (d), the compressed and reduced mixture is then optionally further processed. The mixture is usually firstly classified for this purpose, which can be carried out e.g. with the help of a vibrating screen.

The classified compacted material can then be packaged e.g. in capsules or minipacks, i.e. small pouches (sachets).

It is likewise possible that the classified compacted material is pressed directly into tablets together with customary excipients.

It is likewise also possible that the classified compacted material is subjected to a wet granulation and the granulated material obtained is then pressed into tablets.

The tablets obtained have the advantageous properties that upon contact with aqueous media they disintegrate into particles measuring <2 mm in every direction in space and thus represent a multiple unit dosage form, just like, the classified compacted material.

According to the invention, multiple unit dosage forms are preferred which (a) contain more than 25 wt.-% of particles containing drug and measuring <2 mm in every direction in space or (b) disintegrate in the presence of aqueous media to an extent of more than 25 wt.-% to form particles containing drug and measuring <2 mm in every direction in space.

The advantage of such small particles is that they can pass through the pylorus unhindered, unlike monolithic dosage forms such as conventional tablets.

When preparing tablets according to the invention, customary additives such as lubricants, flow regulators, disintegrants, colorants, plasticizers, anti-adherent agents or binders can also be added to the compacted material. Particularly preferred here are microcrystalline cellulose, magnesium stearate, Aerosil R 972, sodiumcarboxymethyl starch and triethyl citrate.

During the granulation of the compressed and reduced mixture, aqueous granulation can also take place as the active ingredient is protected by the pretreatment with the polymer. Finally, the finished pharmaceutical composition can also still be provided with a rapidly disintegrating film.

An examination of the compressed and reduced mixture reveals particles in which the polymer develops a coherent matrix which leads to the sustained release of the drug. SEM photos accordingly show particles with a homogeneous structure, and particles of the starting material are no longer recognizable. It is assumed that, as a result of the high compression of the mixture which took place in step (b) and the brief increase in temperature introduced via the rollers, a flowing of the polymer around the drug particles results without the polymer entering into a liquid aggregate state.

At any rate it can be established that even without the use of organic solvents and water, pharmaceutical compositions are obtained with the process according to the invention from which the drug is released in a sustained manner, although according to the state of the art the compaction used merely served to improve the flow or increase of density of the compacted material.

It was shown that the compositions obtained according to the invention released the drug over a period of up to 8 hours. However, a composition with rapid release obtained in conventional manner examined under the same conditions already released the drug within an hour.

Finally therefore the invention also relates to a pharmaceutical composition which is obtainable by the process according to the invention.

The composition according to the invention is preferably present in the form of capsules, pouches, styli, tablets or minitablets.

Particularly preferred is a pharmaceutical composition which contains (i) more than 25 wt.-% particles containing drug and measuring <2 mm in every direction in space or (ii) which upon contact with aqueous media disintegrates into more than 25.-% particles containing drug and measuring <2 mm in every direction in space.

A mixture of different compacted materials of the same drug can also be used in order to arrive in this way at a composition according to the invention which has a specific release profile for the drug.

The invention will be explained below in more detail using examples.

### EXAMPLES

### Example 1

### Sustained-release Compacted Material

Formulation:

Oxacarbazepine 30 kg

Ammonium methacrylate copolymer (Eudragit RSPO) 9 kg

The oxcarbazepine is mixed with the ammonium methacrylate copolymer in a high-shear mixer (Diosna P 100) for 5 mins. The mixture was then compressed using a compactor, namely 3-W-Polygran, Gerteis Maschinen+Processengineering AG, Jona, Switzerland (roller width 10 cm and roller speed 7 rpm) with a force of more than 15 and up to 40 kN/cm of roller width after the rollers were heated to 80° C., thermostat-controlled. The resultant ribbon was crushed by means of forced screening (1-mm screen tray), and the compacted material obtained was classified using a vibrating screen (Engelmann screen channel with 0.25 screen tray). The classified compacted material was then packed into hard gelatin capsules of sizes 3, 2, 1 and 0 on a capsule-packing machine. Dosage units of 150 to 300 mg of drug per single dose resulted.

The classified compacted material was also packed into small pouches, also called minipacks or sachets, on a bagging machine. Dosage units of 50 to 2400 mg per single dose thereby resulted.

### Example 2

### Sustained-release Granulated Material

Formulation:

| | |
|---|---|
| Oxcarbazepine | 30 kg |
| Ammonium methacrylate copolymer (Eudragit RSPO) | 9 kg |
| (1^{st} part) | |
| 30- % ammonium methacrylate copolymer dispersion | 2.5 kg |
| in water (Eudragit RS 30 D) | |
| Ammonium methacrylate copolymer (Eudragit RSPO) | 2.5 kg |
| (2^{nd} part) | |
| Talcum | 0.9 kg |
| Triethyl citrate | 0.2 kg |

The oxcarbazepin was firstly mixed with the first part of the ammonium methacrylate copolymer in a high-shear mixer for 5 mins. The mixture was then compacted on a compactor at 20 kN/cm of roller width after the rollers were heated to 80° C. The resulting ribbon was then crushed using forced screening and the resultant compacted material was classified using a vibrating screen. The classified compacted material was then mixed in a fluidized-bed granulator (Glatt WSG 60) with the second part of the ammonium methacrylate copolymer and granulated with the help of the aqueous ammonium methacrylate copolymer dispersion accompanied by the addition of triethyl citrate and talcum.

The granulated material obtained was packed into hard-gelatin capsules of sizes 3, 2, 1 and 0 on a capsule-packing machine. Dosage units of 150 to 300 mg active ingredient per single dose thereby resulted.

The classified compacted material was furthermore packaged in pouches on a bagging machine and dosages of 500 to 2400 mg per individual dose thereby resulted.

### Example 3

### Sustained-release Tablet from Compacted Material

Formulation:

| | |
|---|---|
| Oxcarbazepine | 30 kg |
| Ammonium methacrylate copolymer | 9 kg |
| Sodiumcarboxymethyl starch | 0.2 kg |
| Magnesium stearate | 0.5 kg |
| Microcrystalline cellulose | 10.3 kg |

The oxcarbazepine was mixed with the ammonium methacrylate copolymer in a high-shear mixer for 5 mins. The mixture obtained was then compacted on a roller compacter at 20 kN/cm after the rollers were heated to 80° C. The resultant ribbon was crushed using forced screening and the compacted material obtained was classified using a vibrating screen. The compacted material was then mixed with the sodiumcarboxymethyl starch, the magnesium stearate and the microcrystalline cellulose and pressed into tablets resulting in dosage units between 150 and 600 mg.

### Example 4

### Sustained-release Tablets from Granulated Material

Formulation:

| | |
|---|---|
| Oxcarbazepine | 30 kg |
| Ammonium methacrylate copolymer (1^{st} part) | 9 kg |
| 30- % ammonium methacrylate copolymer dispersion | 2.5 kg |
| Ammonium methacrylate copolymer (2^{nd} part) | 2.5 kg |
| Talcum | 0.9 kg |
| Triethyl citrate | 0.2 kg |
| Sodiumcarboxymethyl starch | 0.2 kg |
| Magnesium stearate | 0.5 kg |
| Microcrystalline cellulose | 5.8 kg |

The oxcarbazepine was mixed with the first part of the ammonium methacrylate copolymer in a high-shear mixer for 5 mins. The mixture was then compacted on a roller compactor at 20 kN/cm of roller width after the rollers were heated to 80° C. The resultant ribbon was crushed using forced screening and the resultant compacted material was classified using a vibrating screen. The classified compacted material was then mixed in a fluidized-bed granulator with the second part of the ammonium methacrylate copolymer and granulated with the aqueous polymer dispersion with the addition of triethyl citrate and talcum.

The granulated material was then mixed with sodium carboxymethyl starch, magnesium stearate and microcrystalline cellulose and pressed into tablets resulting in dosage units between 150 and 600 mg.

1. Process for the preparation of a pharmaceutical composition with sustained drug release wherein
(a) a mixture is provided which contains a drug and a polymer effecting sustained drug release,
(b) the mixture is compressed by passing it between two rollers which have a temperature of more than 40° C. and which exert a force in the range from more than 15 and up to 40 kN/cm of roller width on the mixture, and
(c) the compressed mixture is reduced to the desired particle size.

2. Process according to claim 1, wherein the rollers have a temperature of up to 100° C.

3. Process according to claim 2, wherein the rollers have a temperature in the range from 70 to 90° C.

4. Process according to claim 1, wherein the rollers exert a force on the mixture in the range from 18 to 23 kN/cm of roller width on the mixture.

5. Process according to claim 1, wherein the mixture contains 5 to 90 wt. % drug.

6. Process according to claim 5, wherein the mixture contains 2 to 50 wt. % polymer.

7. Process according to claim 1, wherein the drug is selected from the group consisting of
alpha- or beta-adrenergics;
alpha- or beta-adrenolytics;
analgesics;
antirheumatics;
antiarthritics;
anticholinergics;
anticonvulsants;
antidepressants;
antiparkinson agents;
antipsychotics;
anxiolytics;
dopamine receptor agonists;
antimigraine agents;
neuroleptics;
neuroprotectives;
non-steroid antirheumatics;
nootropics;
sedatives;
hypnotics; and combinations thereof.

8. Process according to claim 7, wherein the drug is an anticonvulsant, an antidepressant, an antiparkinson agent, an antipsychotic, an anxiolytic or a dopamine receptor agonist.

9. Process according to claim 8, wherein the drug is oxcarbazepine, valproic acid or a salt thereof, sulthiame, carbamazepine, lamotrigine or levetiracetam.

10. Process according to claim 1, wherein the polymer is an acrylic acid polymer or acrylic acid copolymer or a salt thereof.

11. Process according to claim 1, wherein the mixture in step (a) also contains at least one substance selected from the group consisting of a colorant, a flow regulator, a lubricant, a dry binder, a disintegrant and a stabilizer.

12. Process according to claim 1, wherein the mixture in step (c) is reduced to a particle size of 50 to 1000 μm.

13. Process according to claim 19, wherein in step (d) the mixture is classified.

14. Process according to claim 19, wherein in step (d) the mixture is packaged in capsules or pouches.

15. Process according to claim 19, wherein in step (d) the mixture is processed to tablets.

16. Pharmaceutical composition which is obtainable by the process of claim 1.

17. Pharmaceutical composition according to claim 16, which is in the form of capsules, pouches, styli, tablets or minitablets.

18. Pharmaceutical composition according to claim 16, which contains (i) more than 25 wt. % particles containing drug and measuring <2 mm in every direction in space or (ii) which upon contact with aqueous media disintegrates into more than 25 wt. % particles containing drug and measuring <2 mm in every direction in space.

19. Process according to claim 1, further including step
(d) further processing the compressed and reduced mixture.

20. Process according to claim 19, wherein in step (d) the mixture is granulated.

21. Process according to claim 20, wherein the granulated mixture is processed to tablets.
